(19) United States
(12) Patent Application Publication
(10) Pub. No.: US 2012/0207705 A1
(43) Pub. Date: Aug. 16, 2012

(54) Stem Cell Conditioned Medium Compositions

(76) Inventor: Bhupendra Vallabh Kara, Cleveland (GB)

(21) Appl. No.: 13/395,779

(22) PCT Filed: Sep. 16, 2010
(86) PCT No.: PCT/GB10/01739

§ 371 Date: Apr. 30, 2012

(30) Foreign Application Priority Data
Sep. 18, 2009 (GB) 0916370.0

(51) Int. Cl. A61K 38/20; A61P 17/02; A61K 38/14; A61K 38/02; A61K 38/39; C12N 5/02; A61K 38/18

(52) U.S. Cl. 424/85.2; 435/404; 514/8.9; 514/9.1; 514/1.1; 514/17.2; 514/20.9

(57) ABSTRACT

A process for preparing a conditioned cell culture medium is provided. The process comprises a) culturing eukaryotic cells in a growth medium having a composition effective to support cell growth; b) separating the cultured cells from the growth medium; and c) maintaining the cultured cells in a basal medium having a composition suitable to maintain cell viability, but not to support substantial cell growth. The cells are preferably dermal sheath, dermal papilla or dermal fibroblast cells. The compositions are useful as pharmaceutical compositions, especially for wound healing.

The present invention relates to compositions for use in pharmaceutical, cosmetic and cosmeceutical applications, particularly wound healing, including the treatment of lesions and burns.

Stem cells are of great interest in numerous therapeutic, cosmetic and cosmeceutical areas because of their capacity to form cells of multiple types. See for example EP 0980270. Additionally, culture media used to grow cells, including stem cells, have been described for therapeutic, cosmetic and cosmeceutical uses arising from the secretion by the growing cells of proteins and other factors into the media. See for example U.S. Pat. No. 7,118,746; U.S. Pat. No. 7,160,726 and WO2008/020815. It remains desirable to identify alternative compositions for therapeutic, cosmetic and cosmeceutical purposes. It is particularly desirable to identify methods that produce conditioned media which are not contaminated with proteinaceous material employed to support growth of the stem cells. Further, it would be particularly desirable to identify effective, scalable methods for the manufacture of conditioned media.

According to a first aspect of the present invention, there is provided a pharmaceutical composition comprising conditioned cell culture medium obtained by a) culturing differentiated human cells retaining stem cell potential selected from the group consisting of dermal sheath cells, dermal fibroblast cells or dermal papilla cells in a growth medium; and b) separating the culture medium from the cells.

Growth media which can be employed in the first aspect of the present invention are culture media sufficient for growth of the cells. Cell culture procedures and culture media are well known in the art, and include basal media supplemented with serum, serum-free media, protein-free media or chemically defined growth media. Growth media typically include essential amino acids, sugars, salts, vitamins, minerals/inorganic salts, trace metals, lipids and nucleosides, and are supplemented with a variety of additional components essential to support cell proliferation such as serum, proteins (for example insulin, transferrin, growth factors and other hormones), antibiotics (for example gentamycin, streptomycin, penicillins), attachment factors (for example fibronectin, collagens, laminins). Supplements can be added in combination, such as in the case of serum, or individually. Growth media provide cells with components necessary to meet the particular cell type's nutritional needs to grow in a controlled in-vitro environment.

In one embodiment, the cell culture process is operated in one culture vessel, the cells are inoculated directly into the culture vessel containing microcarriers, the cells are propagated until the desired cell density is reached. In other embodiments, the cell culture process is operated in at least two distinct cell culture vessels/systems, such as one or more seed expansion vessels followed by the cell production vessel. This multiple seed expansion process preferably employs culture vessels of increasing size until a sufficient number of cells is obtained for the inoculation of the final production cell culture vessel. The seed expansion culture vessels can be of the same type (e.g. tissue culture flasks, shake flasks, roller bottles, spinner flasks, wave bioreactors, stirred tank bioreactors) but increasing in size as the seed expansion progresses or can be a mixture of culture systems increasing in size as the seed culture is expanded in readiness for transfer to the production bioreactor (for example tissue culture flasks to shake flasks to spinner flasks to stirred tank bioreactor systems).

The in-vitro environment is typically controlled to maintain optimum growth temperature, dissolved oxygen, carbon dioxide, pH and osmolality. Many cell culture medium formulations are known in the art or can be obtained readily from commercial sources. It is known to those skilled in the art that conditioned cell culture medium can be produced by seeding cells in growth medium that permits growth of the cells over the period of cell culture. At the end of the cell culture or at a selected point during the culture the cells are removed and the conditioned medium is harvested. The conditioned medium will contain many of the components of the original cell culture growth medium but in addition will also contain cellular metabolites and additional proteins secreted by the cells. Secreted proteins may be biologically active growth factors, cytokines, proteases and other extracellular proteins and peptides. In many embodiments, the composition according to the first aspect of the present invention comprises one or more of Gro-α, I-309, IL-6, IL-8, IL-13, MIF, PAI-1, SDF-1 and TGF-β proteins, especially TGF-β1.

According to a second aspect of the present invention, there is provided a process for preparing a conditioned cell culture medium comprising:

a) culturing eukaryotic cells in a growth medium having a composition effective to support cell growth;
b) separating the cultured cells from the growth medium;
c) maintaining the cultured cells in a basal medium having a composition suitable to maintain cell viability, but not to support substantial cell growth.

Eukaryotic cells which can be employed in the second aspect of the present invention are described in ‘Basic Cell Culture’ Oxford University Press (2002) Ed. J. M. Davis; and ‘Animal Cell Culture’ Oxford University Press (2000) Ed. John. R. W. Masters; both of which are incorporated herein in their entirety by reference. The term “stem cells” describes cells that can give rise to cells of multiple tissue types. Stem cells are cells from the embryo, fetus or adult which have the capacity to become different cell types when presented with specific signaling complexes that provide the directions to do so. There are different types of stems cells. A single totipotent cell is formed when a sperm fertilizes an egg, and has thereby has the capacity to form an entire organism. In the first hours after fertilization, this cell divides into identical totipotent cells. Approximately four days after fertilization and after several cycles of cell division, these totipotent stem cells begin to specialize. When totipotent cells become more specialized, they are then termed “pluripotent.” Pluripotent cells can be differentiated to every cell type in the body, but do not give rise to the placenta, or supporting tissues necessary for foetal development. Because the potential for differentiation of pluripotent cells is not “total,” such cells are not termed “totipotent” and they are not embryos. Pluripotent stem cells undergo further specialization into multipotent stem cells, which are committed to differentiate to cells of a particular lineage specialized for a particular function. Multipotent cells can be differentiated to the cell types found in the tissue from which they were derived; for example multipotent (adult) stem cells such as mesenchymal stem cells, such as dermal sheath, dermal papilla and dermal fibroblast cells.

Cells may be derived from adult, neonatal or foetal tissue and may be autologous or allogenic. The cells may be genetically modified using methods well established in the art. The genetic modification may be used to alter the concentration of one or more component secreted into the cell growth conditioned cell culture medium or the conditioned basal cell culture medium such as, for example, to up or down-regulate a protein, to introduce a new protein, or to regulate ion concentration.

In certain embodiments, the cells are grown as a co-culture. Co-cultured cells are a mixture of two or more different kinds of cells that are grown together.

Cells suitable for use in the process of the second aspect can be obtained by methods known in the art. In particular cells can be isolated from tissues, expanded from cell previously established cell stocks, passaged and cultured to produce the cell growth conditioned cell culture medium or the conditioned basal cell culture medium. The cell growth conditioned cell culture medium or the conditioned basal cell culture medium may be produced using un-differentiated or differentiated cells.

Cells employed in the process of the second aspect of the invention are preferably differentiated human cells retaining stem cell potential selected from the group consisting of dermal sheath cells, dermal fibroblast cells or dermal papilla cells.

Growth media which can be employed in the second aspect of the present invention are as described above in respect of the first aspect. Cells are cultured in growth medium until the desired cell density is achieved.

Basal media employed in the second aspect of the present invention have a composition suitable to maintain cell viability, for example a pH and osmolality to avoid cell lysis, but not to support substantial cell growth, and preferably no cell growth. Basal media comprise basic constituents such as inorganic salts, amino acids, vitamins and an energy source, including sugars, but are not supplemented with components such as serum, proteins, hormones and attachment factors. A preferred energy source comprises glutamine. Basal media are protein-free prior to introduction of the cultured cells, and no protein supplements are added to the basal media after introduction of the cultured cells. The composition of the basal media are selected so as to maintain the viability of the cultured cells to allow export of cellular metabolites and secretions into the basal media. Examples of basal media which may be employed include Ames Medium, Basal Medium Eagle's, Click's Medium, Dulbecco's Modified Eagle's Medium, Ham's Nutrient mixture F-12, Glasgow Minimum Essential Medium, Iscove's Modified Dulbecco's Medium, Minimum Essential Medium Eagle and RPMI-1640 Medium.

In many preferred embodiments of the second aspect of the present invention, the cells are washed after separation from the growth medium, and prior to introduction into the basal medium. Examples of suitable wash solutions for cells are well known in the art, and include buffers, such as phosphate-buffered saline. In some preferred embodiments, the wash solution employed is a basal medium, such as those described above, and commonly the same basal medium in the cells are to be subsequently maintained.

The introduction of cultured cells into the basal medium can be at the same cell concentration achieved at the end of cell growth period or more preferably at a higher concentration to increase the concentration of secreted components into the basal medium. Where the cells are grown in 2D culture, cells are commonly grown to yield a highly confluent monolayer. Such cell concentrations are typically from 1×10⁴ to 1×10⁵ cells per cm², preferably from 2×10⁴ to 5×10⁴ cells per cm². Such highly confluent monolayers of cells are also employed where the contact with the basal medium is conducted in 2D mode. Where the cells are grown in 3D culture, such as attached to microcarriers, cells are commonly grown to concentrations in the range of from 1×10⁷ to 1×10¹² cells per litre, preferably from 1×10⁸ to 1×10¹⁰ cells per litre. In many embodiments, in either 2D or 3D cultures, the volume of basal medium employed is up to 15, commonly from 2 to 10, preferably from 4 to 6, such as about 5, times, lower than the volume of medium employed to support the growth of the cells.

The cultured cells are commonly maintained in the basal medium until the medium has the desired composition, commonly for a period of greater than 12 hours, typically from 18 to 26 hours, such as about 24 hours. At the end of this re-incubation period the cells are removed to generate cell-free conditioned basal cell culture medium. The conditioned basal cell culture medium will contain cellular metabolites and secreted proteins. Secreted proteins may be biologically active growth factors, cytokines, proteases and other extracellular proteins and peptides.

Various terms are used to describe cells in culture. ‘Cell culture’ generally refers to cells taken from a living organism and grown under controlled conditions. A primary cell culture is a culture of cells, tissues or organs taken directly from organisms before the first subculture. Cells are expanded in culture when they are placed in a growth medium under conditions that facilitate growth and/or division, resulting in a larger population of cells. A cell line is a population of cells formed by one or more sub-cultivations of a primary cell culture. Each round of sub-culturing is referred to as a passage. It will be understood by those skilled in the art that there may be many population doublings during the period of passaging.

Anchorage dependant or attachment dependant cells are cells that need to attach to a surface for propagation and growth in tissue culture. In some embodiments, the cells used in carrying out the invention are capable of growing in suspension cultures. As used herein, suspension-competent cells are those that can grow in suspension without making large, firm aggregates, i.e., cells that are mono-disperse or grow in loose aggregates with only a few cells per aggregate. Suspension-competent cells include, without limitation, cells that grow in suspension without adaptation or manipulation and cells that have been made suspension-competent by gradual adaptation of attachment-dependent cells to suspension growth. If such cells are used, the propagation of cells may be done in suspension, thus microcarriers may be used only in the final propagation phase in the production bioreactor itself and in the production phase. In case of suspension-adapted cells, the microcarriers used are typically macroporous carriers wherein the cells are attached by means of physical entrapment inside the internal structure of the carriers.

As used herein, the terms “microcarrier” mean small, discrete particles suitable for cell attachment and growth. Often, although not always, microcarriers are porous beads which are formed from polymers. Microcarriers may also have a dense surface with dents. Usually, cells attach to and grow on the outer surfaces of such beads.

The process of the present invention is carried out by cultivating the cells under conditions conducive to the growth of the cells. Culture conditions, such as temperature, pH, dissolved oxygen (including hypoxic low oxygen conditions) and the like, are those known to be optimal for the particular cell and will be apparent to the skilled person or artisan within this field (see, e.g., Animal Cell Culture: A Practical Approach 2^{nd} Ed., Rickwood, D. and Hames, B. D., eds., Oxford University Press, New York (1992)).

In the first and second aspects of the present invention, the cells are advantageously cultivated attached to a solid support medium. Options for large scale production include tissue culture flasks, roller bottles, perfusion based systems (e.g. hollow fibre bioreactors, internal and external spin filters, acoustic cell retention devices, filtration based cell retention devices) single, multi-plate or stacked-plate cell culture systems, cell cubes, and microcarriers. Cells may also be cultivated using a three dimensional scaffold composed of any material and or shape that allows cells to attach to it and allows cells to grow in one than one layer. The structure of the framework can include a mesh, a sponge or can be formed from a hydrogel. One suitable three dimensional framework is Integra™ Dermal Regeneration Template (Integra Life Sciences). The cells may be cultivated directly on the three dimensional scaffold or may be harvested from tissue culture flasks, roller bottles, hollow fibre systems, single, multi-plate or stacked-plate cell culture systems, cell cubes and microcarriers prior to being re-seeded onto the three dimensional scaffold to produce cell growth conditioned cell culture medium or conditioned basal cell culture medium. Cells maybe also be cultivated using perfusion cell culture. In perfusion cell culture the cells are retained in the bioreactor using a cell retention device such as a filter (e.g. internal or external spin filters), cell retaining mesh, cell settler, acoustic device, etc. Cell culture growth medium is fed continuously or periodically to the bioreactor and cell free ‘spent’ medium is removed continuously or periodically.

In certain preferred embodiments, the cells are attached to the surface of solid microcarriers or attached to, or attached by physical entrapment inside, the internal structure of macroporous microcarriers wherein the microcarrier is a gelatin (hydrolysed collagen) microcarrier. Such microcarriers can comprise gelatin particles, cross linked gelatin particles or gelatin used as a coating on carrier materials such as polystyrene or glass particles. Gelatin can be from a natural source or recombinantly or synthetically produced.

In some embodiments, the cell culture process is operated in one culture vessel. The cells are inoculated directly into the culture vessel containing microcarriers, and the cells are propagated until the desired cell density is reached. The microcarriers containing the propagated cells are aseptically harvested and washed. The washed microcarriers are then resuspended in basal medium and incubated under optimum conditions to maintain cell viability for a period of time (typically 24 hours). The conditioned medium is then harvested. The wash step may be carried out once or multiple times.

In other embodiments, the cell culture process is operated in at least two distinct cell culture vessels/systems, such as one or more seed expansion vessels followed by the cell production vessel. This multiple seed expansion process preferably employs culture vessels of increasing size until a sufficient number of cells is obtained for the inoculation of the final production cell culture vessel. The seed expansion culture vessels can be of the same type (e.g. tissue culture flasks, shake flasks, roller bottles, spinner flasks, wave bioreactors, stirred tank bioreactors) but increasing in size as the seed expansion progresses or can be a mixture of culture systems increasing in size as the seed culture is expanded in readiness for transfer to the production bioreactor (for example tissue culture flasks to shake flasks to spinner flasks to stirred tank bioreactor systems).

Medium exchange can be performed if desired by allowing the microcarriers to settle to the bottom of the cell culture vessel, after which a selected percentage, up to and including all, of the growth medium volume is removed, the microcarrier is optionally washed, and a corresponding percentage of fresh cell culture growth medium is added to the cell culture vessel. The microcarriers are then re-suspended in the medium and culturing continued. This process of medium removal and replacement can be repeated until the desired cell density is achieved.

Gelatin microcarriers which can be employed in the method of the present invention are typically roughly spherical but can have other shapes and can be either porous or solid. Both porous and solid types of microcarriers are commercially available from suppliers. Macroporous gelatin microcarriers are available commercially for example “Cultispher” microcarriers available from Percell Biolytica AB, Sweden. Gelatin macroporous microcarriers are characterised in that the particles are based on a highly cross linked gelatin matrix, particle size of 10-500 μm and consist of a polymer matrix enclosing a large number of cavities having a diameter of 1-50 μm. The use of microcarriers for cell attachment facilitates the use of stirred tank and related bioreactors for the growth of anchorage dependant cells. The cells generally attach to the suspended particles. The desirability of suspensions typically limits the physical parameters of the microcarriers that can be used. Microcarrier particle size range is commonly selected to be large enough to accommodate the anchorage dependant cell type while small enough to form suspensions with properties suitable for use in cell culture bioreactors such as shake flasks, roller bottles, spinner flasks, wave bioreactors and stirred tank bioreactor systems. Gelatins or collagens can be cross-linked via the amine groups of lysine, via carboxyl groups glutamic acid or aspartic acid, or a combination thereof.

Cells are separated from the media in which they have been grown or maintained by methods known in the art for example using, cell settling and decant, batch or continuous centrifugation and/or microfiltration. The cell-free media obtained may be further processed to concentrate or reduce one or more factors or components, for example using ultrafiltration, diafiltration or chromatographic purification.

The conditioned medium produced in the process of the second aspect of the present invention is preferably employed as a pharmaceutical composition. Accordingly, such pharmaceutical compositions form a third aspect of the present invention. The pharmaceutical compositions, especially those derived from dermal sheath cells, dermal fibroblast cells or dermal papilla cells are commonly employed useful in wound and lesion healing. The compositions may also be used for other applications for which the components of the medium are known to be effective.

Preferred compositions comprise one or more of IL-6, Gro-α, SDF-1, FGF-2, SPARC, PAI-1, IL-8, Collagen, Fibronectin, 1-309, IL-13, MIF and SDF-1 and TGF-β proteins, especially TGF-β1. Especially preferred compositions comprise one or more of the proteins listed in Tables 2, 3 or 4.

The compositions of the first and third aspects of the present invention can be employed as pharmaceuticals as liquids or may be frozen, lyophilized, formed into films or dried into a powder. The compositions may be diluted, concentrated, mixed with other components, or be partially or completely purified. The compositions may be delivered to the human or animal body by any suitable means. The conditioned media may be formulated with a pharmaceutically acceptable carrier as a vehicle for internal administration, applied directly to wound/lesion, formulated with a salve or ointment for topical applications, or, for example, made into or added to or dispersed in a biodegradable polymer or hydrogel to create wound dressings, implantable compositions and coatings for medical devices. One advantage of dispersion into a biodegradable polymer is that the system can be used for slow-release delivery systems. This is particularly advantageous to the delivery of bioactive components from the polymer to chronic wounds which must be resistant to rapid degradation from the wounds proteolytic environment and have sustained release of bioactive components. It will be evident to the skilled person that the delivery method will depend on the particular in-vivo application to which the conditioned medium is to be delivered and the skilled person will be able to determine which means to employ accordingly.

Tissues may be regenerated or repaired through the enhancement of endogenous tissue repair by applying secretions from cells instead of or in-addition to the cells. The present invention is based on the premise that multiple complex processes involving the differential expression/secretion of multiple proteins are necessary for optimal tissue repair and re-modelling. The conditioned media produced in the present invention contain many of the regulatory proteins believed to be important in tissue repair, re-modelling and wound healing and which have been shown to depleted for example in, in-vivo models of wound healing. Examples of such proteins include TGF-β, IL-6, Gro-α, SDF-1, FGF-2, SPARC, PAI-1, IL-8, Collagen, Fibronectin, I-309, IL-13, MIF and SDF-1.

TGF-β1 is the predominant TGF-β protein in cutaneous wound healing. In wound healing TGF-β1 is important in inflammation, angiogenesis, re-epithelialisation and connective tissue regeneration. It is shown to have increased expression with the onset of injury (Kopecki Z, Luchetti M M, Adams D H, Strudwick X, Mantamadiotis T, Stoppacciaro A, Gabrielli A, Ramsay R G, Cowin A J, J Pathol 2007; 211:351-61. Kane C J, Hebda P A, Mansbridge J N, Hanawalt P C, J Cell Physiol 1999; 148:157-73.) In-vitro studies have shown that TGF-β1 helps initiate granulation formation by increasing the expression of genes associated with extracellular matrix (ECM) formation including fibronectin, fibronectin receptor, and collagen and protease inhibitors (White L A; Mitchell T I; Brinckerhoff C E, Biochimica et biophysica acta, 2000; 1490 (3):259-68. Mauviel A, Chung K Y, Agarwal A, Tamai K, Uitto J, J Biol Chem 1996; 271:10917-23. Papakonstantinou E, Aletra A J, Roth M, Tamm M, Karakiulakis G, Cytokine 2003, 24: 25-35. Zeng G, McCue H M, Mastrangelo L, Mills A J, Exp Cell Res 1996; 228:271-6). Further in-vitro studies have shown TGF-β1 playing a role in wound contraction by facilitating fibroblast contraction of the collagen matrix (Meckmongkol T T, Harmon R, McKeown-Longo P, Van De Water L, Biochem Biophys Res Commun 2007; 360:709-14). In the matrix formation and re-modelling phase of wound healing, TGF-β1 is involved in collagen production, particularly type I and II (Papakonstantinou E, Aletra A J, Roth M, Tamm M, Karakiulakis G, Cytokine 2003; 24:25-35). When over-expressed, TGF-β1 has been shown to stimulate connective tissue growth factor (CTGF) also known to play an important role in the development of hypertrophic and keloid scars (Colwell A S, Phan T T, Kong W, Longaker M T, Lorenz P H, Plast Reconstr Aesthet Surg 2005; 116:1387-90).

IL-6 has been shown to be important in initiating the wound healing response and expression is increased after wounding, tending to persist in older wounds (Sogabe Y, Abe M, Yokoymana Y, Ishikawa, O. Wound Repair Regen 2006; 14:457-62. Grellner W, Georg T, Wilske J, Forensic Sci Int 2000; 113:251-64. Finnerty C C, Herndon D N, Przkora R, Pereira C T, Oliveira H M, Queiroz D M, Rocha A M, Jeschke M G, Shock 2006; 26:13-9). 11-6 has a mitogenic (Randle M Gallucci, Dusti K Sloan, Julie M Heck, Anne R Murray and Sijy J O'Dell, Journal of Investigative Dermatology (2004) 122, 764-772) and proliferative (Sato M, Sawamura D, Ina S, Yaguchi T, Hanada K, Hashimoto I, Arch Dermatol Res 1999; 291:400-4. Peschen M, Grenz H, Brand-Saberi B, Bunaes M, Simon J C, Schopf E, Vanscheidt W, Arch Dermatol Res 1998; 290:291-7) effect on keratinocytes and is chemoattractive to neutrophils.

Gro-α (CXCL1) chemokine is a member of the CXC family and is a potent regulator of neutrophil chemotaxis and is upregulated in the acute wound. In-vitro studies suggest a role in re-epithelialisation by promoting keratinocyte migration (Englehardt E, Toksoy A, Goebeler M, Debus S, Brocker E B, Gillitzer R, Am J Pathol 1998; 153:1849-60. Christopherson K II, Hromas R, Stem Cells 2001; 19:388-96).

SDF-1 (CXCL12) plays a role in the inflammatory response by recruiting lymphocytes to the wound and promoting angiogenesis. When homeostasis is disturbed in an acute wound, SDF1 is seen at increased levels at the wound margin (Toksoy A, Muller V, Gillitzer R, Goebeler M, Br J Dermatol 2007; 157:1148-54). SDF-1 promotes proliferation and migration of epithelial cells (Salcedo R, Wasserman K, Young H A, Grimm M C, Howard O M, Anver M R, Kleinman H K, Murphy W J, Oppenheim J J, Am J Pathol 1999; 154:1125-35). SDF-1 may also enhance keratinocyte proliferation therefore contributing to re-epithelialisation (Florin L, Maas-Szabowski N, Werner S, Szabowski A, Angel P, J Cell Sci 2005; 118(Pt 9):1981-9).

FGF-2 (bFGF) regulates the synthesis and deposition of various ECM components, increases keratinocyte motility during re-epithelialisation (Sogabe Y, Abe M, Yokoymana Y, Ishikawa, O. Wound Repair Regen 2006; 14:457-62. Grellner W, Georg T, Wilske J, Forensic Sci Int 2000; 113:251-64. Di Vita G, Patti R, D'Agostino P, Caruso G, Arcara M, Buscemi S, Bonventre S, Ferlazzo V, Arcoleo F, Cillari E, Wound Repair Regen 2006; 14:259-64) and promotes migration of fibroblasts and stimulates them to produce collagenase (Sasaki T, J Dermatol. 1992 November; 19(11):664-6).

SPARC (Secreted Protein Acidic and Rich in Cysteine) is expressed in different tissues during re-modelling and repair, such as healing cutaneous wounds (Reed M J, Puolakkainen P, Lane T F, Dickerson D, Bornstein P, Sage E H, J Histochem Cytochem 1993, 41:1467-1477) suggesting a function in regeneration (Louise H. Jørgensen, Stine J. Petersson, Jeeva Sellathurai, Ditte C. Andersen, Susanne Thayssen, Dorte J. Sant, Charlotte H. Jensen and Henrik D. Schroder, Journal of Histochemistry and Cytochemistry, Volume 57 (1): 29-39, 2009). A number of matricellular proteins show increased expression in response to injury (Bradshaw A D, Sage E H, J Clin Invest 2001, 107:1049-1054). SPARC is a matricellular glycoprotein and modulates the interaction of cells with the ECM. Accelerated cutaneous wound closure and altered deposition of collagen have been reported in SPARC-null mice (Bradshaw A D, Reed M J, Sage E H, J Histochem Cytochem 2002, 50:1-10). From expression patterns at the wound site and in-vitro studies, SPARC has been implicated in the control of wound healing (Basu A, Kligman L H, Samulewicz S J, Howe C C, BMC Cell Biol. 2001; 2:15. Epub 2001 August 7).

PAI-1 (SerpineE1) is an important physiological regulator for the generation of plasmin. While PAI-1 is not normally expressed by keratinocytes in the epidermis, it has been shown to be increased in expression following in-vitro and in-vivo wound injury (Romer J, Lund L R, Eriksen J, Ralfkiaer E, Zeheb R, Gelehrter T D, Dano K, Kristensen P, J Invest Dermatol 1991, 97:803-811. Staiano-Coico 1, Carano K, Allan V M, Steiner M G, Pagan-Charry I, Bailey B B, Babaar P, Rigas B, Higgins P J, Exp Cell Res 1996, 227:123-134). A study supporting a role for PAI-1 in wound healing indicates that a loss of PAI-1 function results in accelerated wound healing (Joyce C. Y. Chan, Danielle A. Duszczyszyn, Francis J. Castellino and Victoria A Ploplis, American Journal of Pathology. 2001; 159:1681-1688). Studies have indicated that uPA and PAI-1 are regulated in their expression, both spatially and temporally, during the migration of keratinocytes and connective tissue cells during re-epithelialization, and tissue remodelling associated with wound healing (Romer J, Lund L R, Eriksen J, Ralfkiaer E, Zeheb R, Gelehrter T D, Dano K, Kristensen P, J Invest Dermatol 1991, 97:803-811).

IL-8 expression is increased in acute wounds (E, Toksoy A, Goebeler M, Debus S, Brocker E B, Gillitzer R, Am J Pathol 1998; 153:1849-60) and has been shown to play a role in re-epithelialisation by increasing keratinocyte migration and proliferation (Michel G, Kemeny L, Peter R U, Beetz A, Reid C, Arenberger P, Ruzicka T, FEBS Lett 1992; 305:241-3. Tuschil A, Lam C, Haslberger A, Lindley I, J Invest Dermatol. 1992 September; 99(3):294-8) It also induces the expression of MMPs in leukocytes, stimulating tissue remodelling (Englehardt E, Toksoy A, Goebeler M, Debus S, Brocker E B, Gillitzer R, Am J Pathol 1998; 153:1849-60). It is a strong chemoattractant for neutrophils, thus participating in the inflammatory response (Rennekampff H O, Hansbrough J F, Kiessig V, Doré C, Sticherling M, Schröder J M, J Surg Res. 2000 September; 93(1):41-54). Furthermore, addition of IL-8 in high levels decreases keratinocyte proliferation and collagen lattice contraction by fibroblasts (Iocono J A, Colleran K R, Remick D G, Gillespie B W, Ehrlich H P, Garner W L, Wound Repair Regen. 2000 May-June; 8(3):216-25).

Collagen and Fibronectin—the proliferative phase of wound healing is characterized by angiogenesis, collagen deposition, granulation tissue formation, epithelialization, and wound contraction (Midwood K. S., Williams L. V., and Schwarzbauer J. E. 2004, The International Journal of Biochemistry & Cell Biology 36 (6): 1031-1037). In fibroplasia and granulation tissue formation, fibroblasts grow and form a new ECM by excreting collagen and fibronectin (Midwood K. S., Williams L. V., and Schwarzbauer J.E. 2004, The International Journal of Biochemistry & Cell Biology 36 (6): 1031-1037). Fibroblasts begin entering the wound site two to five days after wounding as the inflammatory phase is ending, and their numbers peak at one to two weeks post-wounding (de la Torre J., Sholar A. (2006), Wound healing: Chronic wounds. Emedicine.com, accessed Jan. 20, 2008). By the end of the first week, fibroblasts are the main cells in the wound (Stadelmann W. K., Digenis A. G. and Tobin G. R. (1998), The American Journal of Surgery 176 (2): 26S-38S). Fibroplasia ends two to four weeks after wounding. In the first two or three days after injury, fibroblasts mainly proliferate and migrate, while later, they are the primary cells that lay down the collagen matrix in the wound site (Stadelmann W. K., Digenis A. G. and Tobin G. R. (1998), The American Journal of Surgery 176 (2): 26S-38S). Fibroblasts from normal tissue migrate into the wound area from its margins. Initially fibroblasts use the fibrin scab formed in the inflammatory phase to migrate across, adhering to fibronectin (Romo T. and Pearson J. M. 2005, Wound Healing, Skin. Emedicine.com, accessed Dec. 27, 2006). Fibroblasts then deposit ground substance into the wound bed, and later collagen, which they can adhere to for migration (Rosenberg L., de la Torre J. (2006), Wound Healing, Growth Factors. Emedicine.com, accessed Jan. 20, 2008). Collagen deposition is considered important because it increases the strength of the wound; before it is laid down, the fibrin-fibronectin clot holds the wound closed (Greenhalgh D. G. (1998), The International Journal of Biochemistry & Cell Biology 30 (9): 1019-1030). Also, cells involved in inflammation, angiogenesis, and connective tissue construction attach to, grow and differentiate on the collagen matrix laid down by fibroblasts (Ruszczak Z. 2003, Advanced Drug Delivery Reviews, 55(12): 1595-1611).

Human cytokine 1-309 is a small glycoprotein, structurally related to a number of inflammatory cytokines, that specifically stimulates human monocytes during angiogenesis (Miller M D, Krangel M S, Proc Natl Acad Sci USA 1992b 89:2950-2954).

In general it is thought desirable in the treatment of wounds to enhance the supply of growth factors by the direct addition of these factors. With this approach the present issues associated with cell based therapy such as but not limited to immune compatibility and tumorgenicity will be eliminated. The cell growth conditioned cell culture medium and the conditioned basal cell culture medium of the present invention is also useful in the treatment of other types of tissue damage wherein the repair and/or regeneration of tissue or damage is desired since many of the array of factors known to be required are found in the applicants' cell growth conditioned cell culture medium and the conditioned basal cell culture medium.

The present invention is illustrated without limitation by the following examples.

### Establishment of Cell Lines

Hair follicle mesenchymal cells were isolated essentially as described in EP980270 with the modifications described below. Human skin tissue samples were washed 3 times with Minimal Essential Medium (MEM, Sigma M4655) containing 1 μg/ml amphotericin and 10 μg/ml gentamycin. Under a dissecting microscope, anagen ‘end bulbs’ were dissected using fine surgical scissors and placed into small volumes (typically 100-200 μl) of MEM. The end bulbs were inverted using needles, and the papilla dissected and the sheath extracted. The papillae and sheaths were then transferred separately to 4 well cell culture plates (Nunc). Ten papillae and 10 sheath were transferred per well in 1 ml of MEM supplemented with 20% foetal bovine serum (FBS), 0.5 μg/ml amphotericin and 5 μg/ml gentamycin. The four well cell culture plates were incubated under sterile and standard conditions (37° C., 5% carbon dioxide). After 10 days cell growth, cells were detached from each well (using standard methods well established in the art) and transferred separately to a 35 mm diameter cell culture dish (Nunc). When cell growth was confluent, the dermal sheath (hereinafter referred to as ‘AVDS’) and dermal papilla (hereinafter referred to as ‘AVDP’) cell lines were detached as previously indicated and transferred to T25 cell culture flasks (Nunc) for further expansion under the conditions described above. Dermal fibroblast (hereinafter referred to as ‘AVDF’) cell lines were established from the same human skin tissue samples described above. The papillary dermis was separated from the reticular dermis and adipose layer and then dissected under a microscope into pieces of approximately 2-3 mm² surface area. Dissected tissue was transferred to a T25 cell culture flask (Nunc) containing MEM supplemented as described for the dermal sheath and dermal papilla cell lines. The T25 cell culture flasks containing dermal fibroblast (AVDF) cell lines were incubated under sterile and standard conditions (as described previously). The dermal fibroblast (AVDF) cell lines were then further expanded using the same conditions when the cultures had reached confluency.

AVDS, AVDP and AVDF cell lines were established form a number of different human tissue samples. A summary of these cell lines which are described in the following examples is provided in Table 1 below.

TABLE 1
Summary of Cell Lines

| Cell Line Designation | Type |
|---|---|
| AVDP2 | Dermal papilla |
| AVDS4 | Dermal sheath |
| AVDS6 | Dermal sheath |
| AVDF3 | Dermal fibroblast |
| AVDF4 | Dermal fibroblast |
| AVDP3 | Dermal Papilla |

### EXAMPLE 1

AVDF4 and AVDS4 cells grown in static culture in MEM+10% FBS were harvested (using standard methods well established in the art). The cells were used to seed 225 cm² flasks at 5×10⁵ cells per flask in 50 ml MEM+10% Foetal Bovine Serum (FBS) and incubated at 37° C., 5% CO₂ for 8 days, with a fresh medium change (MEM+10% FBS) on day 4. The conditioned medium was harvested on day 8, filtered (0.2 μm) and stored frozen at −20° C. prior to analysis.

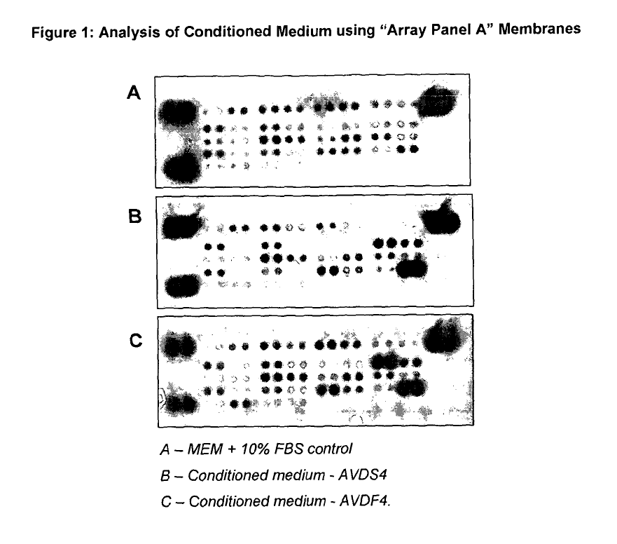

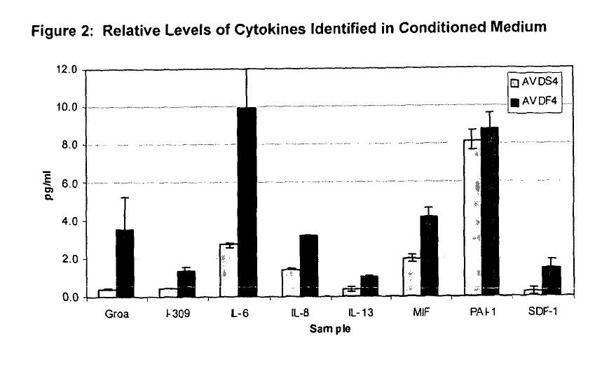

Conditioned medium from AVDF4 and AVDS4, was analysed using a human cytokine array “panel A” kit (R&D Systems ARY005) following the method provided with the kit. MEM+10% FBS was also analysed as a control since AVDF4 and AVDS4 growth medium contained 10% FBS. The results obtained are presented in FIG. 1. The spots identified with increased intensity relative to the MEM+10% FBS control were quantified using methods well described in the art. The data were normalised with respect to the positive controls on each membrane and with respect to the corresponding cytokine spots on the MEM+10% FBS control membrane. Results presented in FIG. 2 present the relative levels of each cytokine identified in AVDS4 and AVDF4 conditioned medium from duplicate samples. Surprisingly amongst the cytokines identified (limited to those included in the “panel A” kit used) cytokines Groα, 1-309, IL6, IL-8 PAI-1 were detected in the conditioned medium produced by both AVDS4 and AVDF4 cell lines. These have been established in the art as being important in facilitating the wound healing process.

### EXAMPLE 2

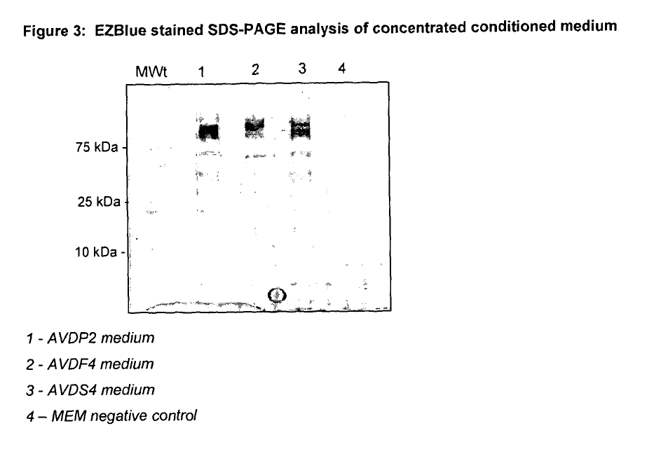

Serum free conditioned medium was prepared from dermal fibroblasts, dermal sheath and dermal papilla cell lines. AVDF4, AVDS4 and AVDP2 cells were grown in static culture in MEM+10% FBS cell culture growth medium at 37° C., 5% CO₂ for 6 days. The cells were harvested (using standard methods well established in the art) and used to separately seed 75 cm² flasks at 2×10⁶ cells per flask in 15 ml of MEM+10% Foetal Bovine Serum (FBS). The flasks were incubated for 24 hours at 37° C., 5% CO₂. After this incubation time the growth medium was removed from each flask and discarded. Cell monolayers were carefully washed three times with 20 ml phosphate buffered saline (PBS) and then a further three times with 20 ml MEM atone (no FBS). Fresh MEM (no FBS, 6 ml) supplemented with 2 mM glutamine was then added to each flask and the flasks incubated for 24 hours at 37° C., 5% CO₂. The conditioned basal medium was harvested from each flask, filtered (0.2 μm) and stored frozen at −20° C. prior to analysis. A sample of MEM growth medium (no FBS) was also included as a control. Samples (8 ml) were thawed and concentrated to 250 μl using Amicon Ultra 15 Centriprep devices (Millipore) at 3200 RCF, 45 min, room temperature, 200 μl of each sample was then concentrated a further 2-fold using a Speedvac. 10 μl of each of the four samples were then analysed using a 4-20% NuPage (Invitrogen) reducing SDS-PAGE gel and stained using EZBIue (Sigma). The SDS-PAGE gel is presented in FIG. 3. Each lane (1-4) of the SDS-PAGE gel was cut with a clean scalpel blade into 10 bands, proteins in each of the 40 bands were digested with trypsin using standard procedures for a ProGest digestion robot. 5 μl of each of the digests were analysed using a Thermo LTQ XL Orbitrap Electrospray mass spectrometer using LC-MS-MS as is well established in the art. MS-MS spectra were searched against a current version of the database Swissprot using Mascot (Matricscience) and Sequest (Thermo) search engines and the ProteomeDiscoverer (Thermo) Interface. Search parameters were set very stringently (FDR<1%). The proteins identified are presented in Tables 2, 3 and 4 for conditioned medium from AVDP2, AVDF4 and AVDS4 respectively.

TABLE 2
Proteins identified in AVDP2 conditioned
basal cell culture medium
Identified proteins

45 kDa calcium-binding protein
6-phosphogluconolactonase
72 kDa type IV collagenase
Abhydrolase domain-containing protein 14B
Adipocyte enhancer-binding protein 1
Alcohol dehydrogenase [NADP+]
Aldose reductase
Alpha-2-macroglobulin
Alpha-actinin-4
Alpha-enolase
Annexin A1
Aspartate aminotransferase, cytoplasmic
ATP synthase subunit beta, mitochondrial
Basement membrane-specific heparan sulfate proteoglycan core protein
Bifunctional aspartokinase/homoserine dehydrogenase 1
Biglycan
Biliverdin reductase A
Cadherin-11
Calsyntenin-1
Calumenin
Carbonyl reductase [NADPH] 1
Cartilage oligomeric matrix protein
Cathepsin B
Cathepsin Z
CD109 antigen
CD166 antigen
Chloride intracellular channel protein 1
Chondroitin sulfate proteoglycan 4
Clusterin
Collagen alpha-1(I) chain
Collagen alpha-1(III) chain
Collagen alpha-1(IV) chain
Collagen alpha-1(V) chain
Collagen alpha-1(VI) chain
Collagen alpha-1(VII) chain
Collagen alpha-1(XI) chain
Collagen alpha-1(XII) chain
Collagen alpha-2(I) chain
Collagen alpha-2(IV) chain
Collagen alpha-2(V) chain
Collagen alpha-2(VI) chain
Collagen alpha-3(VI) chain
Complement C1q tumor necrosis factor-related protein 5
C-type lectin domain family 11 member A
Cystatin-C
Dickkopf-related protein 3
Dihydropyrimidinase-related protein 2
DNA-(apurinic or apyrimidinic site) lyase
EGF-containing fibulin-like extracellular matrix protein 2
Endoplasmic reticulum aminopeptidase 1
Endoplasmic reticulum protein ERp29
Endosialin
Extracellular matrix protein 1
F-actin-capping protein subunit alpha-1
Fascin
Fibrillin-1
Fibronectin
Fibulin-1
Filaggrin-2
Filamin-A
Filamin-B
Filamin-C
FK506-binding protein 9
Fructose-bisphosphate aldolase A
Galectin-1
Galectin-3-binding protein
Gamma-glutamyl hydrolase
Gelsolin
Glia-derived nexin
Glucose-6-phosphate isomerase
Glutathione S-transferase P
Glutathione transferase omega-1
Glyceraldehyde-3-phosphate dehydrogenase
Glycyl-tRNA synthetase
Heat shock 70 kDa protein 6
Heat shock protein beta-1
Heme-binding protein 2
HLA class I histocompatibility antigen, alpha chain G
Hyaluronan and proteoglycan link protein 1
Inactive serine protease RAMP
Insulin-like growth factor-binding protein 2
Insulin-like growth factor-binding protein 3
Insulin-like growth factor-binding protein 4
Insulin-like growth factor-binding protein 6
Insulin-like growth factor-binding protein 7
Integrin beta-like protein 1
Interstitial collagenase
Keratin, type I cytoskeletal 9
Kinesin-like protein KIF7
Lactoylglutathione lyase
Lamin-A/C
Laminin subunit beta-1
Latent-transforming growth factor beta-binding protein 2
Latent-transforming growth factor beta-binding protein, isoform 1S
Leucine-rich repeat-containing protein 15
Leucine-rich repeat-containing protein 40
L-lactate dehydrogenase A chain
L-lactate dehydrogenase B chain
Lumican
Lysyl oxidase homolog 2
Malate dehydrogenase, cytoplasmic
Malate dehydrogenase, mitochondrial
Mammalian ependymin-related protein 1
Matrix-remodeling-associated protein 8
Metalloproteinase inhibitor 1
Mitochondrial fission factor homolog A
Moesin
Neuropilin-1
Nucleobindin-1
Olfactomedin-like protein 2B
Olfactomedin-like protein 3
Out at first protein homolog
Peptidyl-prolyl cis-trans isomerase B
Peroxidasin homolog
Peroxiredoxin-1
Peroxiredoxin-4
Peroxiredoxin-6
Phosphatidylethanolamine-binding protein 1
Phosphoglycerate kinase 1
Phosphoglycerate mutase 1
Phosphoserine aminotransferase
Pigment epithelium-derived factor
Plasminogen activator inhibitor 1
Plectin-1
Polypeptide N-acetylgalactosaminyltransferase 10
Polypeptide N-acetylgalactosaminyltransferase 2
Polypeptide N-acetylgalactosaminyltransferase 5
Pregnancy-specific beta-1-glycoprotein 5
Procollagen C-endopeptidase enhancer 1
Procollagen-lysine,2-oxoglutarate 5-dioxygenase 1
Procollagen-lysine,2-oxoglutarate 5-dioxygenase 2
Procollagen-lysine,2-oxoglutarate 5-dioxygenase 3
Profilin-1
Protein disulfide-isomerase A6
Protein S100-A6
Purine nucleoside phosphorylase
Putative heterogeneous nuclear ribonucleoprotein
A1-like protein 3
Putative nucleoside diphosphate kinase
Putative quinone oxidoreductase
Pyruvate kinase isozymes M1/M2
Quinone oxidoreductase
Rab GDP dissociation inhibitor alpha
Rab GDP dissociation inhibitor beta
Ras GTPase-activating-like protein IQGAP1
Reticulocalbin-1
Rho GDP-dissociation inhibitor 1
Scavenger receptor cysteine-rich domain-containing protein LOC284297
Semaphorin-7A
Serine protease HTRA1
Serpin B6
Serpin B7
Serum albumin
S-formylglutathione hydrolase
Stanniocalcin-2
Stromelysin-1
Sulfhydryl oxidase 1
Target of Nesh-SH3
Thioredoxin domain-containing protein 5
Thioredoxin-dependent peroxide reductase,
mitochondrial
Thrombospondin-1
Transaldolase
Transforming growth factor-beta-induced protein ig-h3
Transketolase
Trypsin-3
Tryptophanyl-tRNA synthetase, cytoplasmic
Ubiquinone biosynthesis protein COQ7 homolog
Ubiquitin carboxyl-terminal hydrolase isozyme L1
Ubiquitin-like modifier-activating enzyme 1
Uncharacterized metallophosphoesterase CSTP1
Urokinase-type plasminogen activator
Vasorin
Versican core protein
Vimentin
Vinculin
WD repeat-containing protein 1
Xaa-Pro dipeptidase
Zinc finger protein 276

TABLE 3
Proteins identified in AVDF4 conditioned
basal cell culture medium
Identified proteins

2′,3′-cyclic-nucleotide 2′-phosphodiesterase
45 kDa calcium-binding protein
60 kDa heat shock protein, mitochondrial
72 kDa type IV collagenase
Aggrecan core protein
Aldose reductase
Alpha-2-macroglobulin
Alpha-actinin-4
Alpha-enolase
Arylsulfatase B
Aspartate aminotransferase, mitochondrial
ATPase family AAA domain-containing protein 5
Band 3 anion transport protein
Basement membrane-specific heparan sulfate proteoglycan core protein
Biglycan
Biotinidase
Calsyntenin-1
Calumenin
Cartilage oligomeric matrix protein
Cathepsin B
Cathepsin Z
CD109 antigen
Collagen alpha-1(I) chain
Collagen alpha-1(III) chain
Collagen alpha-1(IV) chain
Collagen alpha-1(V) chain
Collagen alpha-1(VI) chain
Collagen alpha-1(XII) chain
Collagen alpha-2(I) chain
Collagen alpha-2(V) chain
Collagen alpha-2(VI) chain
Collagen alpha-3(IV) chain
Collagen alpha-3(VI) chain
Collagen triple helix repeat-containing protein 1
Complement C1r subcomponent
Complement C1s subcomponent
Dickkopf-related protein 3
Dihydrolipoyl dehydrogenase, mitochondrial
Dihydropyrimidinase-related protein 2
EGF-containing fibulin-like extracellular matrix protein 2
Endoplasmic reticulum protein ERp29
Extracellular matrix protein 1
Fascin
Fibrillin-1
Fibronectin
Fibulin-1
Filamin-A
Filamin-B
Filamin-C
FK506-binding protein 10
Fructose-bisphosphate aldolase A
Galectin-3-binding protein
Gamma-glutamyl hydrolase
Gelsolin
Glucose-6-phosphate isomerase
Heat shock protein beta-1
Immunoglobulin superfamily containing leucine-rich repeat protein
Inactive serine protease RAMP
Insulin-like growth factor-binding protein 4
Insulin-like growth factor-binding protein 6
Insulin-like growth factor-binding protein 7
Integrin beta-like protein 1
Interstitial collagenase
Laminin subunit alpha-4
Laminin subunit beta-1
Laminin subunit gamma-1
Latent-transforming growth factor beta-binding protein 2
Latent-transforming growth factor beta-binding protein, isoform 1S
L-lactate dehydrogenase A chain
Lumican
Lysyl oxidase homolog 2
Macrophage metalloelastase
Malate dehydrogenase, cytoplasmic
Matrix-remodeling-associated protein 5
Matrix-remodeling-associated protein 8
Metalloproteinase inhibitor 1
Moesin
Nidogen-1
Olfactomedin-like protein 3
Olfactory receptor 5C1
Out at first protein homolog
Peptidyl-prolyl cis-trans isomerase B
Periostin
Peroxiredoxin-1
Phosphoglycerate kinase 1
Phosphoglycerate mutase 1
Phosphoserine aminotransferase
Pigment epithelium-derived factor
Plasminogen activator inhibitor 1
Plastin-3
Pregnancy-specific beta-1-glycoprotein 5
Procollagen C-endopeptidase enhancer 1
Procollagen-lysine,2-oxoglutarate 5-dioxygenase 1
Procollagen-lysine,2-oxoglutarate 5-dioxygenase 2
Procollagen-lysine,2-oxoglutarate 5-dioxygenase 3
Profilin-1
Prolyl 3-hydroxylase 1
Protein BCCIP homolog
Protein disulfide-isomerase A6
Protein ZNF750
Putative heterogeneous nuclear ribonucleoprotein
A1-like protein 3
Putative nucleoside diphosphate kinase
Pyruvate kinase isozymes M1/M2
Rab GDP dissociation inhibitor alpha
Rab GDP dissociation inhibitor beta
Ras GTPase-activating-like protein IQGAP1
Reticulocalbin-1
Semaphorin-7A
Serine protease HTRA1
Serpin B6
Serpin B7
Stanniocalcin-2
Stromelysin-1
Sulfhydryl oxidase 1
Testican-1
Thioredoxin domain-containing protein 5
Thrombospondin-1
Thrombospondin-2
Transaldolase
Transforming growth factor-beta-induced protein ig-h3
Transgelin
Transketolase
Tryptophanyl-tRNA synthetase, cytoplasmic
Tyrosine-protein kinase receptor UFO
Ubiquitin carboxyl-terminal hydrolase isozyme L1
Ubiquitin-like modifier-activating enzyme 1
Vasorin
Vimentin
Vinculin
Vitronectin
WD repeat-containing protein 1

TABLE 4
Proteins identified in AVDS4 conditioned
basal cell culture medium
Identified proteins

45 kDa calcium-binding protein
60 kDa heat shock protein, mitochondrial
72 kDa type IV collagenase
78 kDa glucose-regulated protein
Adenylyl cyclase-associated protein 1
Aldose reductase
Alpha-2-macroglobulin
Alpha-actinin-4
Alpha-enolase
Alpha-N-acetylglucosaminidase
Aspartate aminotransferase, cytoplasmic
Aspartate aminotransferase, mitochondrial
Band 3 anion transport protein
Basement membrane-specific heparan sulfate proteoglycan core protein
Beta-1,4-galactosyltransferase 5
Biglycan
Biotinidase
Cadherin-11
Cadherin-2
Calsyntenin-1
Calumenin
Cartilage oligomeric matrix protein
Cathepsin B
Cathepsin D
Cathepsin Z
CD109 antigen
CD166 antigen
CD44 antigen
Chloride intracellular channel protein 1
Collagen alpha-1(I) chain
Collagen alpha-1(III) chain
Collagen alpha-1(IV) chain
Collagen alpha-1(V) chain
Collagen alpha-1(VI) chain
Collagen alpha-1(VII) chain
Collagen alpha-1(X1) chain
Collagen alpha-1(XII) chain
Collagen alpha-2(I) chain
Collagen alpha-2(IV) chain
Collagen alpha-2(V) chain
Collagen alpha-2(VI) chain
Collagen alpha-3(VI) chain
Complement C1r subcomponent
C-type lectin domain family 11 member A
Cyclin-A2
Cystatin-C
Decorin
Dickkopf-related protein 3
Dihydrolipoyl dehydrogenase, mitochondrial
Dipeptidyl-peptidase 3
DnaJ homolog subfamily A member 4
Dystroglycan
EGF-containing fibulin-like extracellular matrix protein 2
Elongation factor 1-gamma
Endoplasmic reticulum protein ERp29
Endosialin
Extracellular matrix protein 1
Ezrin
Fascin
Fibrillin-1
Fibrillin-2
Fibronectin
Fibulin-1
Filamin-A
Filamin-B
Filamin-C
Fructose-bisphosphate aldolase A
Fructose-bisphosphate aldolase C
Galectin-1
Galectin-3-binding protein
Gamma-glutamyl hydrolase
Gelsolin
Glia-derived nexin
Glucose-6-phosphate isomerase
Glutathione S-transferase P
Glutathione transferase omega-1
Glyceraldehyde-3-phosphate dehydrogenase
Glycyl-tRNA synthetase
G-protein coupled receptor 143
Heat shock 70 kDa protein 6
Heat shock protein beta-1
Hyaluronan and proteoglycan link protein 1
Ig gamma-1 chain C region
Immunoglobulin superfamily containing
leucine-rich repeat protein
Inhibin beta A chain
Insulin-like growth factor-binding protein 4
Insulin-like growth factor-binding protein 6
Insulin-like growth factor-binding protein 7
Integrin beta-like protein 1
Interstitial collagenase
Lamin-A/C
Latent-transforming growth factor beta-binding protein 2
Latent-transforming growth factor beta-binding protein, isoform 1S
Legumain
Leucine-rich repeat-containing protein 15
L-lactate dehydrogenase A chain
Lumican
Lysyl oxidase homolog 2
Macrophage mannose receptor 2
Macrophage migration inhibitory factor
Malate dehydrogenase, cytoplasmic
Malate dehydrogenase, mitochondrial
Matrix-remodeling-associated protein 5
Metalloproteinase inhibitor 1
Microfibrillar-associated protein 2
Microfibrillar-associated protein 5
Moesin
Moesin/ezrin/radixin homolog 1
Neuropilin-1
Nidogen-1
Nuclear receptor coactivator 5
Nucleobindin-1
Nucleobindin-2
Olfactomedin-like protein 3
Peptidyl-prolyl cis-trans isomerase B
Periostin
Peroxidasin homolog
Peroxiredoxin-1
Peroxiredoxin-6
Phosphoglycerate kinase 1
Phosphoglycerate mutase 1
Phosphoserine aminotransferase
Pigment epithelium-derived factor
Plasminogen activator inhibitor 1
Platelet-derived growth factor D
Plectin-1
Polypeptide N-acetylgalactosaminyltransferase 10
Polypeptide N-acetylgalactosaminyltransferase 2
Procollagen C-endopeptidase enhancer 1
Procollagen-lysine,2-oxoglutarate 5-dioxygenase 1
Procollagen-lysine,2-oxoglutarate 5-dioxygenase 2
Procollagen-lysine,2-oxoglutarate 5-dioxygenase 3
Profilin-1
Prolyl 3-hydroxylase 1
Protein disulfide-isomerase A6
Protein ZNF750
Putative heterogeneous nuclear ribonucleoprotein
A1-like protein 3
Putative nucleoside diphosphate kinase
Pyruvate kinase isozymes
M1/M2
Rab GDP dissociation inhibitor alpha
Rab GDP dissociation inhibitor beta
Ras GTPase-activating-like protein IQGAP1
Reticulocalbin-1
Semaphorin-7A
Serine protease HTRA1
Serpin B6
Serpin B7
Stanniocalcin-2
Stromelysin-1
Sulfhydryl oxidase 1
Testican-1
Thioredoxin domain-containing protein 5
Thrombospondin-1
Thrombospondin-2
Transaldolase
Transforming growth factor-beta-induced protein ig-h3
Transgelin
Transketolase
Tryptophanyl-tRNA synthetase, cytoplasmic
Tyrosine-protein kinase receptor UFO
Ubiquitin carboxyl-terminal hydrolase isozyme L1
Ubiquitin-like modifier-activating enzyme 1
Vasorin
Vimentin
Vinculin
Vitronectin
WD repeat-containing protein 1

Surprisingly a total of 177 different human proteins were identified in conditioned basal cell culture medium from cell line AVDP2, 131 different human proteins in conditioned basal medium from cell line AVDF4 and 167 different human proteins in conditioned basal medium from cell line AVDS4. This was particularly un-expected given the relatively short incubation period and basal cell culture medium used to produce the conditioned basal cell culture medium. The cells described in this document can therefore be used as sources of any or all of these proteins, or any proteins or other molecules which are secreted or expressed by them. Those skilled in the art will appreciate the analysis indicates the presence of proteins well established in the art as important for wound healing and will appreciate that new proteins have been identified. It will be evident to those skilled in the art how the differences between the three cell lines could be used to produce different physical embodiments of the conditioned basal cell culture medium or how the conditioned basal cell culture medium may be further processed to concentrate or reduce one or more factors or components, for example using ultrafiltration, diafiltration or chromatographic purification.

### EXAMPLE 3

A 225 cm² cell culture flask (Nunc) of dermal fibroblast cells AVDF3 grown in static culture conditions were detached and cell number determined using methods well described in the art. 2.3×10⁶ cells were used to seed a 1.5 L cell culture spinner flask containing 1.5 g/L CultiSpher S microcarriers (prepared as described by the manufacturer) in a total volume of 330 ml of serum free growth medium supplemented with 2 mM glutamine (Sigma). The headspace of the spinner flask was equilibrated with 5% CO₂, 2% O₂ gas. The spinner flask was transferred to a cell culture incubator at 37° C. and agitated at 35 rpm using a magnetic stirrer base. After 4 days incubation under the conditions described 35 ml of cell culture supernatant was removed from the spinner flask and replaced with fresh serum free growth medium (as described above). On day 5 and day 7 incubation, 50 ml of culture supernatant was removed and replaced with fresh serum free medium as described above. On day 8, 80 ml of culture supernatant was removed and replaced with fresh serum free medium. After a total of 10 days in culture the AVDF3 cells were detached from the microcarrriers using methods well established in the art. 1.35×10⁷ cells were used to inoculate a glass cell culture bioreactor (Applikon) in a total volume of 2 L of serum free growth medium supplemented with 2 mM glutamine, 0.2% Pluronic F-68 and 1.5 g/L Cultispher S microcarriers (prepared as described previously). The bioreactor was cultured at a temperature of 36.5° C., pH 7.0 (manual control by carbon dioxide gas sparging and/or addition of sodium hydroxide), dissolved oxygen tension 5.0% (air saturation) and an agitator speed of 40 rpm which was increased gradually to 60 rpm over the course of the culture. The dissolved oxygen level in the cell culture was maintained using CO₂ and N₂ gas sparging. Emulsion C antifoam agent (Sigma) was added to the bioreactor when foaming was observed. After 4 days incubation under the conditions described, 200 ml of culture supernatant was removed from the bioreactor and replaced with fresh serum free growth medium (as described above). A further 200 ml of culture supernatant was removed and replaced with fresh serum free growth medium (as described above) on days 6, 8, 10, 11, 13, 15, 17 and 21. After 17 days of growth, 200 ml of culture (medium and microcarriers with cells attached) was harvested aseptically. The harvested culture was aliquoted equally into four 50 ml conical sample tubes and the microcarriers with cells attached were allowed to sediment to the base of the sample tube under gravity. Microcarrier free culture medium was carefully removed and the sedimented microcarriers with cells attached were washed first 3× with PBS and a further 3× with MEM (no FBS) to remove traces of spent growth medium from the original cell culture. The microcarriers with cells attached were pooled into a final volume of 45 ml of MEM (no FBS)+2 mM glutamine. This suspension was used to seed 3×E125 shake flasks, with approximately 2×10⁶ cells per flask. The headspace of the flask was equilibrated with 5% CO₂, 2% O₂ gas and transferred to an orbital shaker for 24 hours at 37° C., 60 rpm. The conditioned basal medium was harvested from each flask, filtered using a 0.2 μm filter, and stored at −20° C. prior to analysis.

### EXAMPLE 4

Two 225 cm² cell culture flasks (Nunc) of dermal fibroblast cells AVDP3 grown in static culture conditions were detached and counted using methods well described in the art. 2.4×10⁶ cells were used to seed a 1.5 L cell culture spinner flask containing 1.5 g/L CultiSpher S microcarriers (prepared as described previously) in a total volume of 300 ml of MesenPro growth medium (low serum, Invitrogen) supplemented with 4 mM glutamine. The headspace of the spinner flask was equilibrated with 5% CO₂, 2% O₂ gas. The spinner flask was transferred to a cell culture incubator at 37° C. and agitated at 35 rpm using a magnetic stirrer base. After 3 days incubation under the conditions described 80 ml of cell culture supernatant was removed from the spinner flask and replaced with fresh growth medium (as described above). After a total of 9 days incubation under the conditions described, a 10 ml sample was taken from the spinner flask and cell number and cell viability determined using standard methods well described in the art. The cell number and cell viability was used to estimate the total viable cell number retained in the spinner flask attached to the microcarriers. The microcarriers with cells attached from the spinner flask were washed using PBS as described previously then suspended in 75 ml MEM (no FBS) and used to seed one 250 ml shake flask with approximately 2.5×10⁷ cells attached to microcarriers. The headspace of the flask was equilibrated with 5% CO₂, 2% O₂ gas and transferred to an orbital shaker for 24 hours at 37° C., 60 rpm. The conditioned basal cell culture medium was harvested from the flask, filtered using a 0.2 μm filter, and stored at −20° C. prior to analysis.

Two 225 cm² cell culture flasks (Nunc) of dermal fibroblast cells AVDS6 grown in static culture conditions were detached and counted using methods well described in the art. 2.4×10⁶ cells were used to seed a 1.5 L cell culture spinner flask containing 1.5 g/L CultiSpher S microcarriers (prepared as described previously) in a total volume of 300 ml of MesenPro growth medium (Invitrogen) supplemented with 4 mM glutamine. The headspace of the flask was equilibrated with 5% CO₂, 2% O₂ gas. The spinner flask was transferred to a cell culture incubator at 37° C. and agitated at 35 rpm using a magnetic stirrer base. After 3 days incubation under the conditions described 80 ml of cell culture supernatant was removed from the spinner flask and replaced with fresh growth medium (as described above). After a total of 9 days incubation under the conditions described, a 10 ml sample was taken from the spinner flask and cell number and cell viability determined using standard methods well described in the art. The cell number and cell viability was used to estimate the total viable cell number retained in the spinner flask. The microcarriers with cells attached were washed using PBS as described previously then pooled in a final volume of 54 ml MEM (no FBS) and used to seed one 250 ml shake flask with approximately 1.8×10⁷ cells attached to microcarriers. The headspace of the flask was equilibrated with 5% CO₂, 2% O₂ gas and transferred to an orbital shaker for 24 hours at 37° C., 60 rpm. The conditioned basal medium was harvested from the flask, filtered using a 0.2 μm filter, and stored at −20° C. prior to analysis.

### EXAMPLE 5

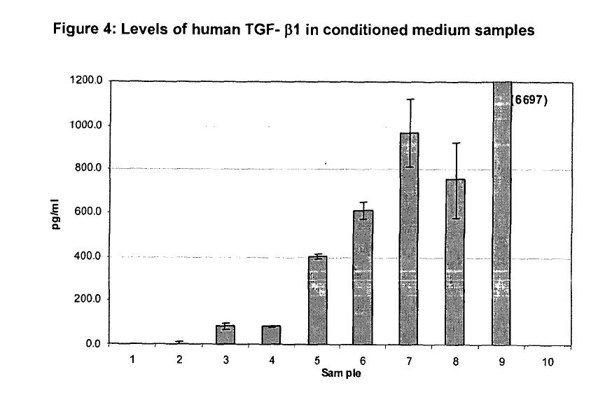

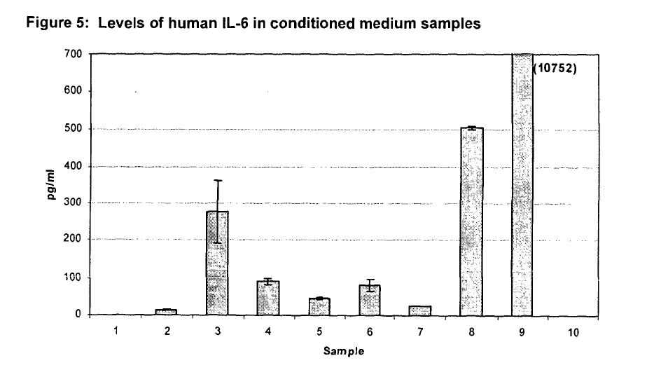

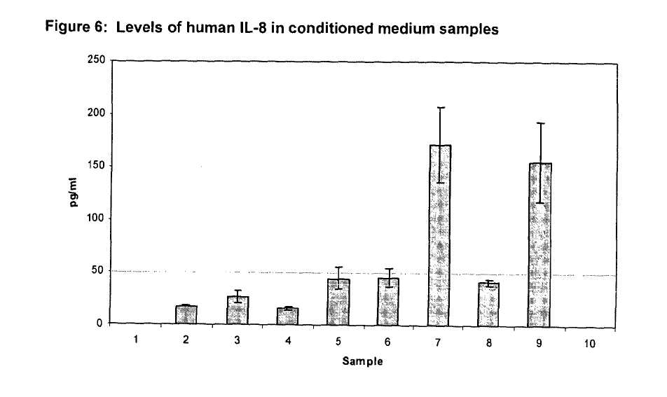

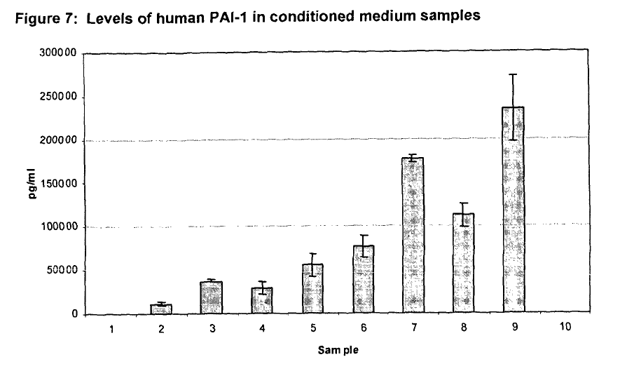

Conditioned medium from AVDF4, AVDS4 and AVDP2 (prepared as described in Examples 1 and 2), AVDF3 (prepared as described in Example 3) and AVDS6 and AVDP3 (prepared as described in Example 4) were analysed using SearchLight Array Technology (Aushon Biosystems Inc.). MEM and MEM+10% FBS growth media were included as controls. Aushon SearchLight Protein Array Technology is a multiplexing sandwich-ELISA system based on chemiluminescent or fluorescent detection of analytes whose respective capture-antibodies are spotted in arrays within each well of a 96-well microplate. Up to 16 analytes (4×4 array in each well) can be measured per well, thus 16 cytokines or other biomarkers can be assayed simultaneously with each sample (50 μl). Samples were assayed for TGFβ-1, IL-6, IL-8 and PAI-1 levels. The concentrations of each protein identified in the conditioned medium/conditioned basal medium samples are presented in FIGS. 4, 5, 6 and 7. Samples are designated numbers 1 to 10 and the identity of these samples is shown in Table 5.

TABLE 5
Sample Identities

| Sample | | Conditioned medium |
|---|---|---|
| number | Cell line designation | production method |
| 1 | MEM control | — |
| 2 | AVDF3 | Example 3 |
| 3 | AVDS6 | Example 4 |
| 4 | AVDP3 | Example 4 |
| 5 | AVDS4 | Example 1 |
| 6 | AVDF4 | Example 1 |
| 7 | AVDP2 | Example 2 |
| 8 | AVDS4 | Example 2 |
| 9 | AVDF4 | Example 2 |
| 10 | MEM + 10% FBS control | — |

The data presented in FIGS. 4-7 exemplify that key proteins involved in wound healing (TGFβ-1, IL-6, IL-8 and PAI-1) can be detected and quantified in conditioned media from the three novel cell types AVDS, AVDP and AVDF. The levels of proteins in the conditioned cell culture medium or conditioned basal cell culture medium can be varied by adjusting the cell concentration used and/or the growth medium composition and/or the cell culture system. It will be also be evident to those with skill in the art how further development of the cell culture growth conditions, cell line used can be carried out to increase cell number attached to the microcarriers and how this will influence secretion of proteins when producing conditioned basal cell culture medium. It will be evident to those skilled in the art that the data generated using the 2 L stirred tank cell culture bioreactor using cells attached to microcarriers demonstrates production of conditioned basal medium that is a scaleable and economic manufacturing system for the large scale production of the physical embodiments of the present invention. It will be apparent to those skilled in the art that on completion of a microcarrier based bioreactor process to expand cells that microcarriers can be sedimented, conditioned medium harvested and/or cells attached to microcarriers washed in-situ and incubated with basal cell culture medium. The conditioned basal medium can be easily be harvested by in-situ sedimentation of the microcarriers (gravity sedimentation) and decanting of cell free conditioned basal cell culture medium.

### EXAMPLE 6

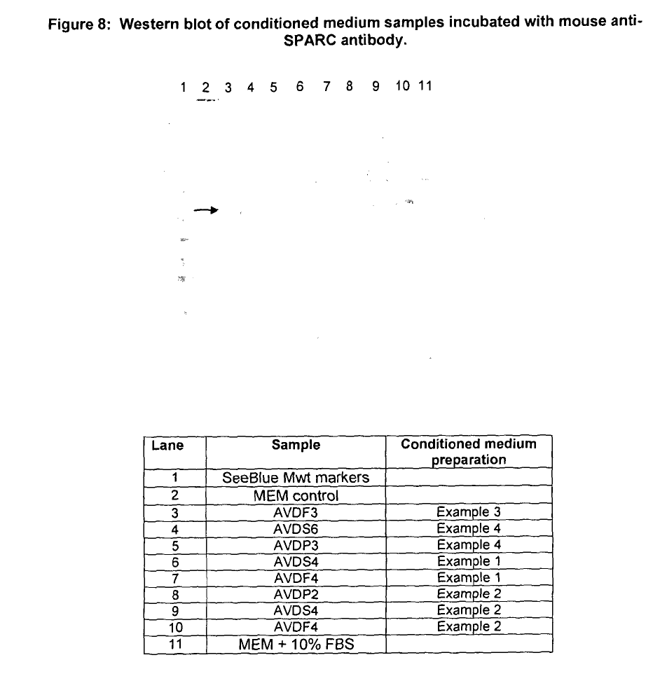

Conditioned medium from AVDF4, AVDS4 and AVDP2 (prepared as described in Examples 1 and 2), AVDF3 (prepared as described in Example 3) and AVDS6 and AVDP3 (prepared as described in Example 4) were analysed by western blotting as is well established in the art. Briefly, samples were reduced and run on 4-12% BisTris gels (Invitrogen) with SeeBlue molecular weight marker (Invitrogen) using MES running buffer. The samples were then transferred to PVDF membranes. After blotting, the membrane was incubated in 15 ml of blocking buffer (PBS+1% BSA) on a rocking platform for 1 hour at room temperature. The blocking buffer was decanted and the membrane was incubated in 8 ml of 1/2000 dilution of mouse monoclonal anti-SPARC antibody (Sigma WH0006678M2) at 4° C. overnight. The membrane was then washed three times in 15 ml PBS+0.05% Tween 20 each for 5 minutes at room temperature on a rocking platform. 8 ml of 1/10,000 dilution of rabbit anti-mouse IgG (whole molecule)-peroxidase (Sigma A9044) was added and incubated at room temperature on a rocking platform for 1 hour. The membrane was then washed three times in 15 ml PBS+0.05% Tween 20 each for 5 minutes at room temperature on a rocking platform. The membrane was developed by incubating in SIGMAFAST™ 3,3′-Diaminobenzidine tablets (Sigma D4418) dissolved in 15 ml of water for 15 minutes at room temperature on a rocking platform. The results of the western blot is presented in FIG. 8.

Conditioned media from all three cell lines AVDS, AVDP and AVDF expanded using cell culture flasks, spinner flask and bioreactor indicate the secretion and accumulation of SPARC protein.

### EXAMPLE 7

Conditioned media from AVDF4, AVDS4 and AVDP2 (prepared as described in Examples 1 and 2), AVDF3 (prepared as described in Example 3) and AVDS6 and AVDP3 (prepared as described in Example 4) were analysed by dot blot (as is well established in the art) for secretion and accumulation of fibronectin and collagen proteins. The PVDF membranes were wetted with methanol and then soaked in PBS, 10 μl of each sample and controls were spotted onto on the membranes. 50 ng and 5 ng of standard (fibronectin—Sigma F1141 or collagen—Sigma C8919) was also spotted onto the membrane and all spots were allowed to air dry for 1 hour. The membranes were then wetted again in methanol, rinsed in PBS and then incubated overnight in 8 ml of blocking buffer (PBS+1% BSA) at 4° C. The blocking buffer was decanted and the membranes were incubated in 4 ml of 1/200 dilution of either mouse monoclonal anti-fibronectin antibody (Sigma F7387) or mouse monoclonal anti-collagen antibody (Sigma C2456) for 2 hours at room temperature on a rocking platform. The membranes were then washed three times in 8 ml PBS+0.05% Tween 20 each for 5 minutes at room temperature on a rocking platform. 4 ml of 1/2000 dilution of rabbit anti-mouse IgG (whole molecule)-peroxidase (Sigma A9044) was added and incubated at room temperature on a rocking platform for 1 hour. The membranes were then washed three times in 8 ml PBS+0.05% Tween 20 each for 5 minutes at room temperature on a rocking platform. The membranes were developed by incubating in SIGMAFAST™ 3,3′-Diaminobenzidine tablets (Sigma D4418) dissolved in 15 ml of water for 15 minutes at room temperature on a rocking platform. The results of the dot blots are presented in Table 6.

Conditioned media from all three cell lines AVDS, AVDP and AVDF expanded using cell culture flasks, spinner flask and bioreactor indicate the secretion and accumulation of collagen protein.

TABLE 6
Analysis of cell growth conditioned cell culture medium and conditioned
basal cell culture medium for the presence of collagen and fibronectin

| | |
|---|---|
| Conditioned | Spot intensity relative to |
| medium | 50 ng standard (+++) |

| Sample | preparation | Collagen | Fibronectin |
|---|---|---|---|
| 50 ng standard | — | +++ | +++ |
| MEM control | — | ND | ND |
| AVDS4 | Example 1 | + | +++ |
| AVDF4 | Example 1 | + | ++++ |
| AVDP2 | Example 1 | ++ | ++++ |
| AVDS4 | Example 2 | (1) | ++ |
| AVDF4 | Example 2 | + | ++++ |
| AVDF3 | Example 3 | ++++ | ++++ |
| AVDS6 | Example 4 | ++ | +++ |
| AVDP3 | Example 4 | +++ | +++ |
| MEM + 10% FBS | | ND | + |

ND = not detected
(1) = dot masked by FBS in sample. LC-MS-MS analysis (Example 2) indicates secretion and accumulation of collagen.

1. A process for preparing a conditioned cell culture medium comprising:
(a) culturing eukaryotic cells in a growth medium having a composition effective to support cell growth;
(b) separating the cultured cells from the growth medium; and
(c) maintaining the cultured cells in a basal medium having a composition suitable to maintain cell viability, but not to support substantial cell growth.

2. A process according to claim 1, where the cells are dermal sheath, dermal papilla or dermal fibroblast cells.

3. A process according to claim 1, wherein the basal medium is protein-free prior to introduction of the cultured cells.

4. A process according to claim 1, wherein the cells are cultured attached to a microcarrier.

5. A process according to claim 1, wherein the cells are washed after separation from the growth medium and prior to introduction into the basal medium.

6. A process according to claim 1, wherein the product of step (c) is subsequently subjected to one or more of the following processes:
(a) partial or complete purification;
(b) freezing;
(c) lyophilisation; and
(d) drying.

7. A composition produced by a process according to claim 1.

8. A pharmaceutical composition produced by a process according to claim 1.

9. A composition according to claim 8 which comprises one or more of IL-6, Gro-α, SDF-1, FGF-2, SPARC, PAI-1, IL-8, Collagen, Fibronectin, I-309, IL-13, MIF and SDF-1 and TGF-βproteins.

10. A method of healing a wound which comprises applying to the wound a pharmaceutical composition according to claim 8.

11. A pharmaceutical composition comprising conditioned cell culture medium obtained by (a) culturing differentiated human cells retaining stem cell potential selected from the group consisting of dermal sheath cells, dermal fibroblast cells or dermal papilla cells in a growth medium; and (b) separating the culture medium from the cells.

12. A composition according to claim 11 which comprises one or more of Gro-α, I-309, IL-6, IL-8, IL-13, MIF, PAI-1, SDF-1 and TGF-β proteins.

13. A composition according to claim 11, which has been further subjected to one or more of the following processes:
(a) partial or complete purification;
(b) freezing;
(c) lyophilisation; and
(d) drying.

14. A method of healing a wound which comprises applying the wound a composition according to claim 11.

15. A process according to claim 6, wherein:
(a) the cells are dermal sheath, dermal papilla or dermal fibroblast cells;
(b) the basal medium is protein-free prior to introduction of the cultured cells;
(c) the cells are cultured attached to a microcarrier; and/or
(d) the cells are washed after separation from the growth medium and prior to introduction into the basal medium

16. A pharmaceutical composition produced by a process according to claim 15.

17. A pharmaceutical composition according to claim 16 which comprises one or more of IL-6, Gro-α, SDF-1, FGF-2, SPARC, PAI-1, IL-8, Collagen, Fibronectin, I-309, IL-13, MIF and SDF-1 and TGF-β proteins.

18. A method of healing a wound which comprises applying to the wound a pharmaceutical composition according to claim 17.

19. A method of healing a wound which comprises applying to the wound a composition according to claim 12.

20. A method according to claim 19, wherein the composition has been further subjected to one or more of the following processes:
(a) partial or complete purification;
(b) freezing;
(c) lyophilisation; and
(d) drying.
